(19) United States
(12) Patent Application Publication
(10) Pub. No.: US 2020/0008788 A1
(43) Pub. Date: Jan. 9, 2020

(54) DEVICE AND METHOD FOR SEALING A MEMBRANE

(71) Applicant: UNIVERSITAET ZÜRICH, Zurich (CH)

(72) Inventors: Yannick DEVAUD, Fribourg (CH); Vincent MILLERET, Zurich (CH); Roland ZIMMERMANN, Gockhausen (CH); Martin EHRBAR, Wil SG (CH); Nicole OCHSENBEIN, Zurich (CH)

(73) Assignee: UNIVERSITAET ZÜRICH, Zurich (CH)

(21) Appl. No.: 16/483,445

(22) PCT Filed: Feb. 5, 2018
(86) PCT No.: PCT/EP2018/052765

§ 371 Date: Aug. 5, 2019

(30) Foreign Application Priority Data
Feb. 6, 2017 (EP) 17154743.3
Sep. 29, 2017 (EP) 17194141.2
Oct. 6, 2017 (EP) 17195183.3

(51) Int. Cl. A61B 17/00; A61L 31/00

(52) U.S. Cl. CPC A61B 17/0057; A61B 17/00491; A61L 31/005; A61B 2017/00597; A61B 2017/0061; A61B 2017/00623; A61B 2017/00787; A61B 2017/0065; A61B 2017/00659; A61B 2017/00867; A61B 2017/00884; A61B 2017/00778; A61B 2017/00628

(57) ABSTRACT

Disclosed embodiments relate to a membrane closure device (100) for closing a perforation in a membrane (10), comprising: a patch guiding mechanism (110) for guiding at least one expandable patch (120) to a perforation site (12) of a membrane (10); and a fastener delivery mechanism (130, 330) for delivering a fastener (140) to the perforation site (12) and for fastening the at least one expandable patch by the fastener (140) to the membrane (10) to seal the perforation in the membrane (10) by the at least one expandable patch (120).

### TECHNICAL FIELD AND BACKGROUND

Embodiments disclosed herein relate in general to devices and methods for sealing a membrane such as a fetal membrane.

The fetal membrane is a relatively thin tissue layer having a thickness of a few hundred microns that surrounds the fetus during development. The fetal membrane holds amniotic fluid and creates a physical barrier, for example, to protect the fetus from infections and to provide paracrine signaling between the mother and the embryo. Intact fetal membranes are of central importance to a pregnancy. Preterm pre-labor rupture of fetal membranes (PPROM) initiates delivery in most cases, which is a serious complication in early pregnancy. Indeed, preterm birth carries a big risk of complication ranging from incurable diseases decreasing life quality and/or expectancy and range from cerebral palsy, impaired cognitive skills, vision problems, hearing problems, behavioral, psychological as well as chronic health issues to morbidity.

With advances in fetal diagnosis and therapies, fetoscopy has become a clinical routine. This surgical technique aims at treating potentially life-threatening diseases during pregnancy by invasive intervention into the amniotic cavity. Example medical indications include twin-to-twin transfusion syndrome (TTTS), discordant monochorionic twins with a lethal anomaly, reverse arterial perfusion (TRAP) as well as severe congenital diaphragmatic hernia (CDH). Depending on the type of intervention, the injury or perforation created by the fetoscopy can induce iatrogenic PPROM (iPPROM) in 6 to 45% of the cases. This unsolved clinical problem compromises benefits of the surgical intervention and blocks tremendously the advances in treatment technologies.

Since fetal membranes have a limited spontaneous healing potential, the application of natural and synthetic materials to close the defect has been studied in the past. Such approaches have failed to prove clinical relevance due to inadequate material stability or to the absence of healing-inducing factors.

The description above is presented as a general overview of related art in this field and should not be construed as an admission that any of the information it contains constitutes prior art against the present patent application.

### GENERAL DESCRIPTION OF THE INVENTION

Embodiments of the present invention are aimed at overcoming some of the disadvantages associated with the application of, e.g., polymeric biomimetic gluing material such as “mussel glue” for the sealing of fetal membranes. The potential in the use of such polymeric biomimetic gluing material for the sealing of membranes was described in Lee et al., Nature 2007, 448. 338-41, and further in Bilic et al., American Journal of Obstetrics and Gynecology 2010, 202. 85 e1-9. Further, Haller et al. disclose in Acta biomaterialia 2012, 8. 4365-70 that mussel glue is non-toxic, tightly adheres to fetal membranes and withstands substantial deformation and/or pressure of fetal membranes.

However, some of the problems that were encountered in all the above-noted studies was the difficulty to deliver the gluing material to the desired location and the lack of a reproducible and robust method to apply the gluing material at the desired location for closing the perforated membrane.

Devices and methods according to embodiments of the present invention may overcome at least some of the above-noted problems.

The following description of devices and methods of the invention is given with reference to particular examples, with the understanding that such devices and methods are not limited to these examples.

The disclosed embodiments relate to membrane closure devices and methods for repairing a perforated (also: ruptured) membrane. Such membrane can, for example, include a fetal membrane, vascular membrane, cardiovascular membrane and/or a tympanic membrane. In conjunction with fetal membranes, perforation may be inflicted on purpose, e.g., in order to conduct fetoscopy. Breakage of the fetal membrane following prior fetoscopy or otherwise invasive procedure is referred to as iatrogenic previable premature rupture of membranes (or I-PPROM).

Non-medical applications may for example include the sealing of membranes in industrial applications or environments such as automotive, process engineering, construction, precision mechanics, and/or the like. Correspondingly, while embodiments disclosed herein relate to repairing living tissue membrane, this should by no means be construed limiting. Accordingly, the devices and methods described herein may be applicable to any type of perforated membrane or layer, whether living or non-living.

According to some embodiments, the membrane closure device is configured or adapted to allow, for example, targeted delivering of glue to patch for the instantaneous gluing thereof to a fetal membrane, without requiring additional mechanical fasteners or elements such as tackers, clamps, staplers, sutures and/or the like, which could perforate, puncture, rupture, or otherwise damage the membrane. The devices and methods of the present invention allow the sealing of a membrane with a single patch. The methods may thus include the delivery and/or use of a single patch for the sealing of a membrane from its underside. Embodiments of the devices and methods allow one-sided sealing of a membrane.

In an embodiment, the patch does not require an adhesive layer, since a glue is provided separate from the patch for fastening thereof to the membrane. In other words, the patch may be free of an adhesive layer during its delivery to the perforation site. Optionally, the glue may comprise a bioactive agent. Optionally, the patch may comprise a bioactive agent. Optionally, only the glue may comprise a bioactive agent.

According to some embodiments, the membrane closure device for closing a perforation in a membrane, comprises a patch delivery mechanism for delivering and deploying one or more (e.g., expandable) patches to a perforation site of the membrane. The membrane closure device further comprises a fastener delivery mechanism for delivering a fastener to the perforation site for fastening the one or more expandable patches, to the membrane to seal the perforation in the membrane. Such fastener can be, for example, a sealant, a sealant-inducing material, healing-inducing matter, a plug, and/or a weld. The thickness of the patch can range, for example, from about 50 micron to about 1 mm. Optionally, the fastener delivery mechanism may include the patch delivery mechanism, or vice versa. Optionally, at least a portion of the fastener delivery mechanism may constitute the patch delivery mechanism, or vice versa.

According to some embodiments, the fastener delivery mechanism comprises one or more syringes barrel operable to receive glue and which are further operable to receive one or more respective plungers for pushing the glue or glue components through corresponding one or more distal ends of the one or more syringe barrel into a retention cavity formed by the patch when in the expanded configuration.

The membrane closure device may be operable to access the perforation site from a proximal side thereof for sealantly securing the patch with glue to the underside or distal side of the perforation site. The membrane closure device may be operable to sealantly secure the patch to the underside of the perforation site without requiring the use of tackers, staples and/or other elements that would puncture or otherwise damage the fetal membrane. In other words, membrane closure device allows securing the patch to the perforation in a “tacker/staple-free manner”. Moreover, the membrane closure device allows the user to operably position the patch without requiring a field-of-view (FOV) of the perforation from the membrane underside, so that, for example, access for fetoscopy and for delivering the patch may be gained via the same perforation. Optionally, no imaging techniques (e.g., Ultrasound imager) may have to be employed while applying the patch to sealantly secure the patch to the perforation site. Hence, only one perforation may be needed to allow conducting fetoscopy and to seal the underside of the perforation by employing the membrane closure device. For example, the membrane closure device was inserted immediately after fetoscopy through the exact same catheter used for the intervention in order to ensure precise placement at the site of puncture. Aspects of embodiments may pertain to a fetoscope that is configured to comprise the membrane closure device.

Optionally, one or more expandable bodies (e.g., balloons) may be employed to allow stabilizing the fetal membrane between such expandable body and the expanded patch while pulling the patch against the fetal membrane. The one or more expandable bodies may allow applying a counterforce in a distal direction while, at the same time, pulling the patch against the fetal membrane in a proximal direction. The membrane closure device may be operable such that the counterforce increases responsive to an increase in the pulling force. The patch and/or the expandable units are selectively expandable.

Merely to simplify the discussion that follows, without be construed limiting, the terms “fastener”, “glue”, and “sealant” may herein be used interchangeably.

The membrane closure device comprises a tube-shaped body (also: tube) having a distal and a proximal end. The tube-shaped body defines internal to the tube a patch delivery passageway (also: operating channel) for delivering the expandable patch in a narrow (e.g., crimped or folded) configuration, via the tube, from the proximal to the distal end for attaching the expandable patch, using the fastener, in an expanded configuration to a surface (e.g., distal surface) of the membrane.

The relative positional term “proximal” as used herein refers to a location that is situated closer from a user of the device during normal use of the device than a location that is “distal” to the user during such normal use. For example, a proximal end is an end point of the tube situated closest to the user of the device, and a distal end is an endpoint situated farthest from the user.

An outer membrane surface of a membrane is a surface that has to be engaged first by the device for gaining access to, e.g., an amniotic cavity enclosed by the membrane. An inner membrane surface may refer to the surface that is in direct contact with the content of, e.g., the amniotic cavity and that can be engaged only after engagement of the device with the outer surface. It is noted that the outer and inner membrane surfaces do not necessarily have to pertain to amniotic membranes, but may also pertain to other membranes or tissue of, e.g., mammals.

Merely to simplify the discussion that follows, the term “proximal membrane surface” may herein also be referred to as “outer surface” or “upper surface”. Correspondingly, the term “inner membrane surface” may herein also be referred to as “inner surface” or “undersurface”.

In the context of the present disclosure, a longitudinal direction of the tube extends from its proximal end along a longitudinal axis to the distal end. The latitudinal direction is perpendicular to the longitudinal axis, along a latitudinal axis of the tube.

When in the folded configuration, the patch can slide (e.g., by applying a pushing and/or pulling force) through the tube along its longitudinal axis from the distal to the proximal end. Upon exiting the tube's distal end, the patch is set in the expanded configuration (e.g., by expanding in latitudinal direction) for covering the perforation.

The term “user” as used herein may include, for example, a physician or medical doctor. The term “patient” as used herein refers to the person that is subjected to procedures for closing a perforated membrane in the body of the patient.

The terms cited above denote also grammatical variations thereof.

Merely, for brevity and clarity, and therefore without be construed limiting, in the present disclosure a membrane closure device is herein exemplified for use during a minimally invasive surgery (MIS).

The outer diameter of the portions of the tube-shaped body perforating the membrane for, e.g., fetoscopy—which may be performed at a gestational stage ranging, e.g., from week 17 to week 27—may range, for example, from 1.7 mm to 4 mm. The diameter may depend, inter alia, on the fetoscopy procedure required to be performed. Fetoscopy procedures can include, for example, placental laser surgery, delivering a balloon into the trachea to occlude the airway for the treatment of congenital diaphragmatic hernia, shunting, and/or any other fetal treatment. The patch in the folded configuration is adapted or configured to slidably fit into the patch delivery passageway, which is internal to the tube-shaped body. In the expanded configuration, the patch's diameter can for example be at least 2, 3, 4, or 5 times larger than the tube-shaped body's outer diameter. The patch's diameter, in the expanded configuration, may for example range from 5 mm to 30 mm).

The patch, when affixed or fastened to the membrane, may be adapted or configured to withstand, e.g., until birth in case of fetoscopy, a certain range of shear forces and pressure that may be imparted in a direction normal to the surface of the patch. For example, amniotic fluid pressure range that may act or be imparted normal to the patch's surface may range from 9 mmHg at gestational stage of week 10 to 5 mmHg at 30 weeks. In TTTS, the amniotic pressure that the patch has to withstand until birth can also go up to 20 mmHg.

The figures illustrate generally, by way of example, but not by way of limitation, various embodiments of the devices and/or methods discussed in the present document.

For simplicity and clarity of illustration, elements shown in the figures have not necessarily been drawn to scale. For example, the dimensions of some of the elements may be exaggerated relative to other elements for clarity of presentation. The number of elements shown in the Figures should by no means be construed as limiting and is for illustrative purposes only. Furthermore, reference numerals may be repeated among the figures to indicate corresponding or analogous elements. References to previously presented elements are implied without necessarily further citing the drawing or description in which they appear. The figures are listed below.

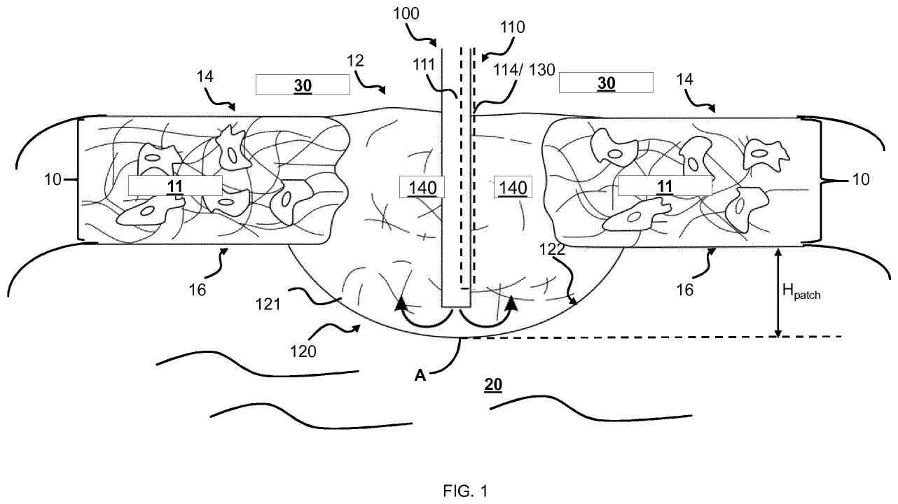

FIG. 1 is a schematic cross-sectional view of a portion of a ruptured amniotic layer sealed with a sealant, according to some embodiments;

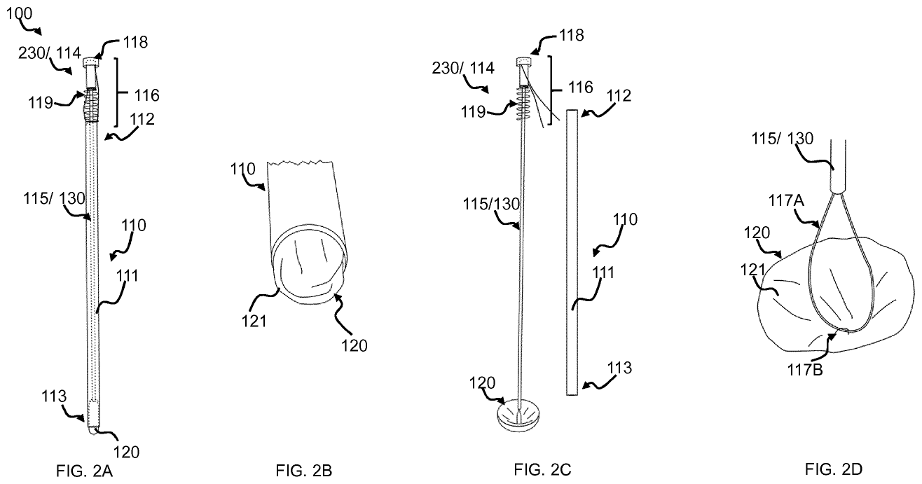

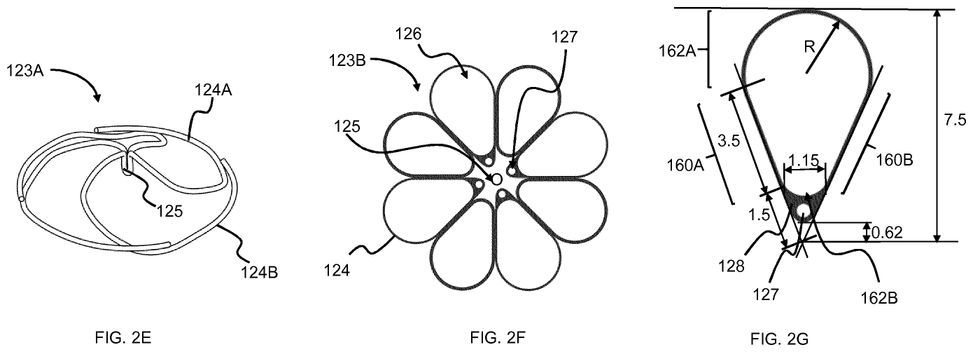

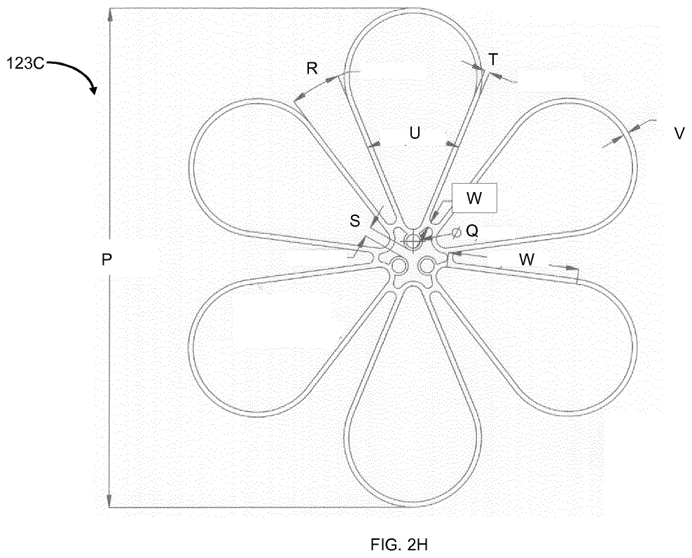

FIG. 2A is a schematic assembly illustration of a membrane closure device, according to some embodiments;

FIG. 2B is a schematic illustration of a patch in a folded configuration located at the distal end of the membrane closure device, according to some embodiments;

FIG. 2C is a schematic disassembly illustration of the membrane closure device, according to some embodiments;

FIG. 2D is a schematic illustration of the patch in an expanded configuration after delivery through the distal end of the membrane closure device, according to some embodiments;

FIG. 2E is a schematic illustration of a support structure of a patch, according to an embodiment;

FIG. 2F is a schematic illustration a support structure of a patch, according to an alternative embodiment;

FIG. 2G is a schematic illustration of example measures of a petal or lobe of the support structure of FIG. 2F, according to some embodiments;

FIG. 2H is a schematic illustration of a support structure, according to some other embodiments;

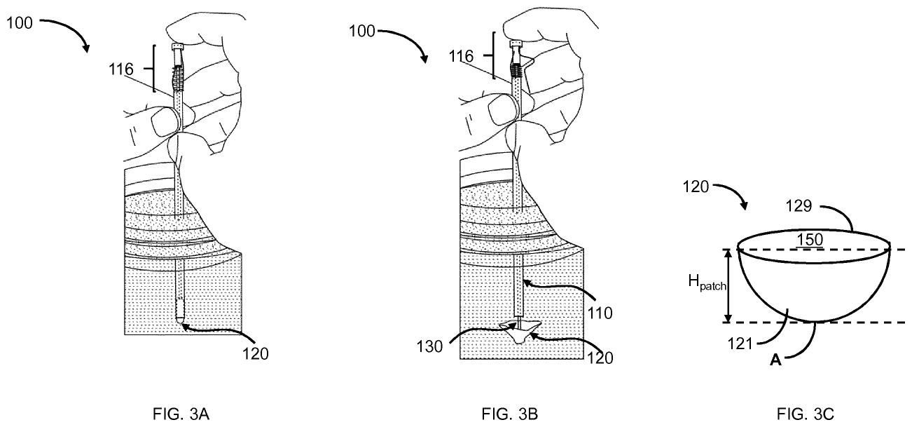

FIGS. 3A and 3B are schematic illustration showing a method of using the membrane closure device, according to some embodiments;

FIG. 3C is a schematic illustration of the patch in an expanded configuration, according to some embodiments;

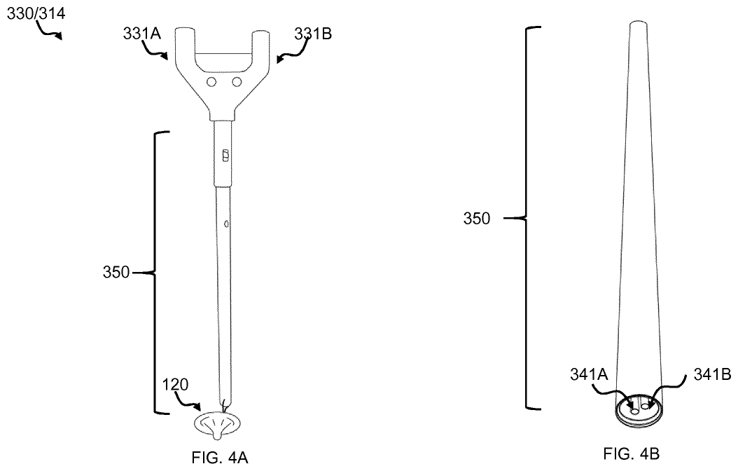

FIG. 4A is a schematic illustration of a fastener delivery mechanism of the membrane closure device, according to some other embodiments;

FIG. 4B is a schematic illustration of a distal end of the fastener delivery mechanism of FIG. 4A;

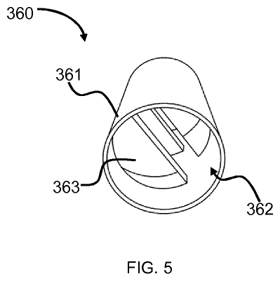

FIG. 5 is a schematic illustration of a mixing chamber of the fastener delivery mechanism, according to some embodiments;

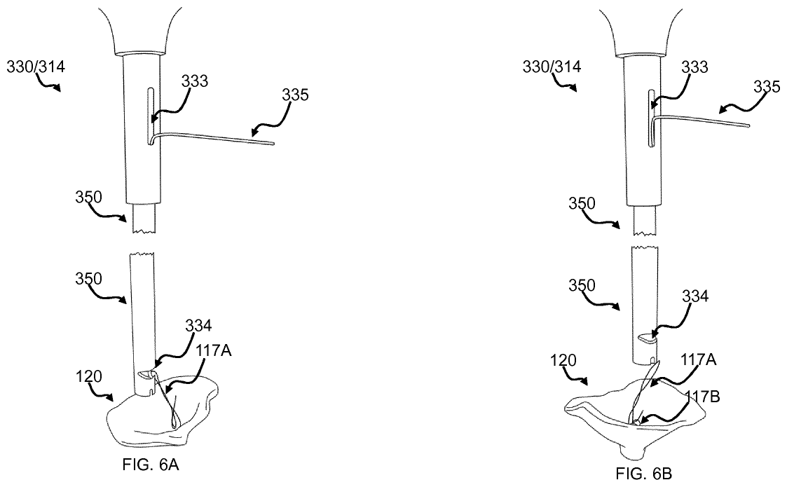

FIG. 6A is a schematic illustration showing the patch in a retained state, according to some embodiments;

FIG. 6B is a schematic illustration showing the patch in a released state, according to some embodiments;

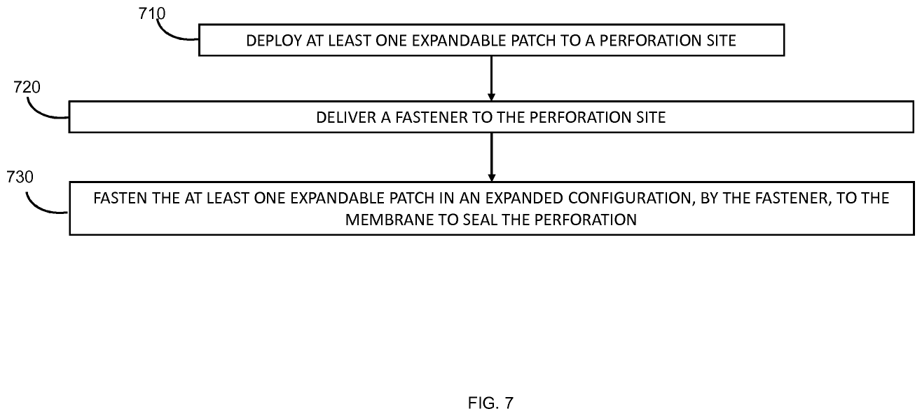

FIG. 7 is a flowchart diagram of a method for closing a perforated membrane, according to some embodiments;

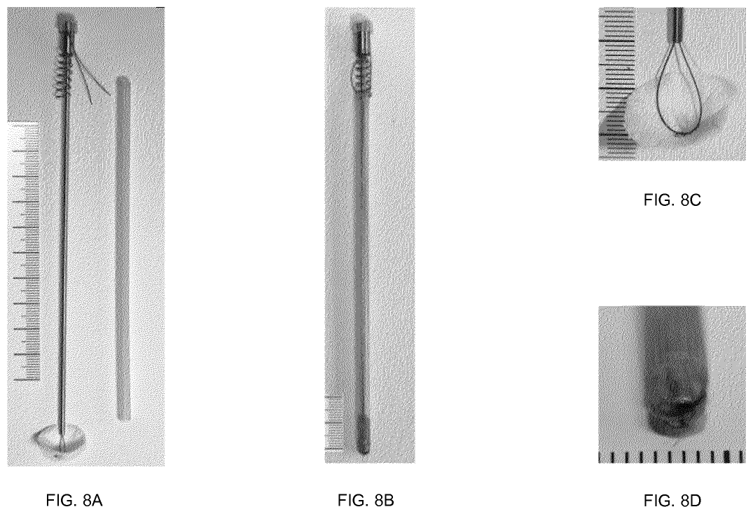

FIGS. 8A to 8D show images of an example implementation of a membrane closure device;

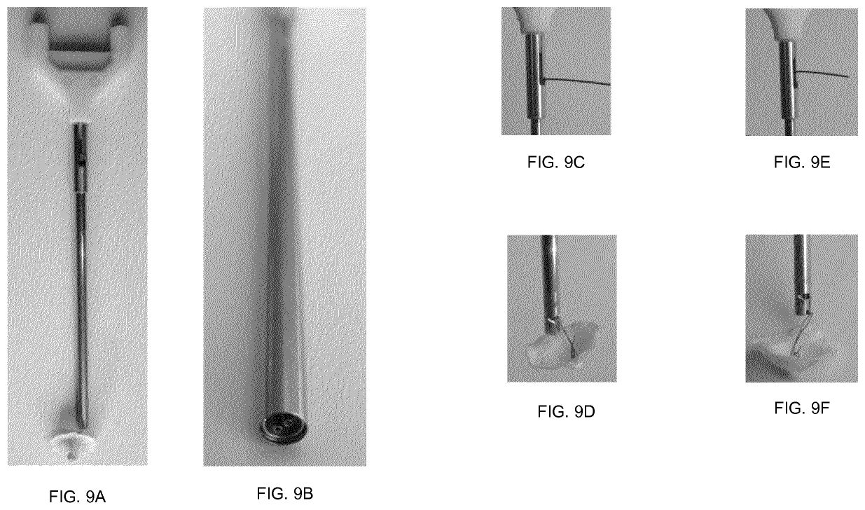

FIGS. 9A to 9F show images of another example implementation of a membrane closure device;

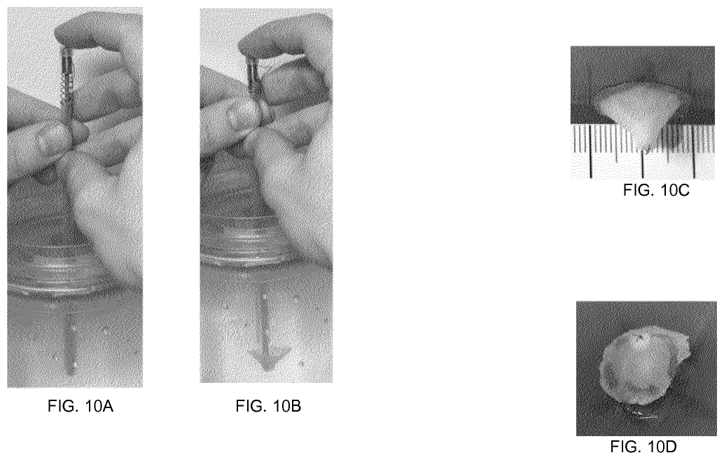

FIGS. 10A to 10D show images of an example in-vitro application using the membrane closure device shown in FIGS. 8A to 8D;

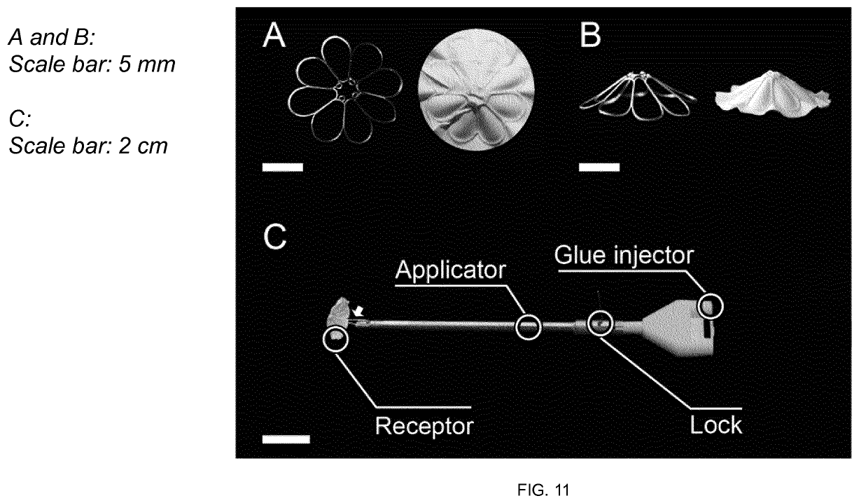

FIGS. 11A and 11B show top and side view photograph of an umbrella-shaped receptor without (left) and with (right) Degrapol® membrane coverage, as used in the experiments;

FIG. 11C shows the applicator prototype (also: membrane closure device prototype) in its expanded configuration, as used in the experiments;

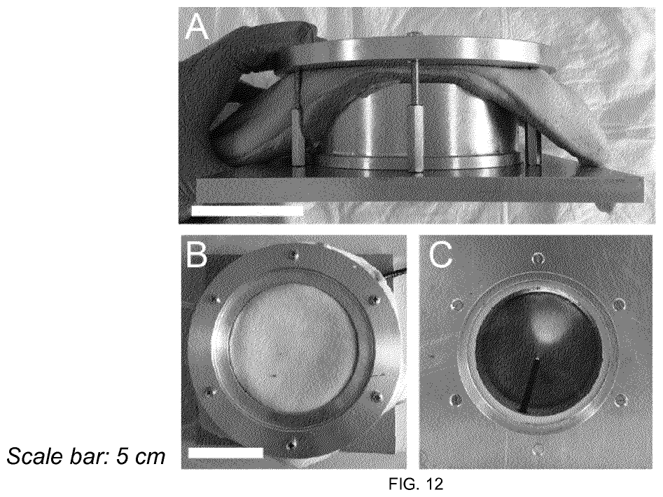

FIG. 12A shows a side view image of an ex vivo fetal membrane model clamped in an aluminum cylinder;

FIG. 12B shows a top view image of the tissue clamped in the aluminum cylinder;

FIG. 12C shows a bottom view of the tissue clamped in the aluminum cylinder;

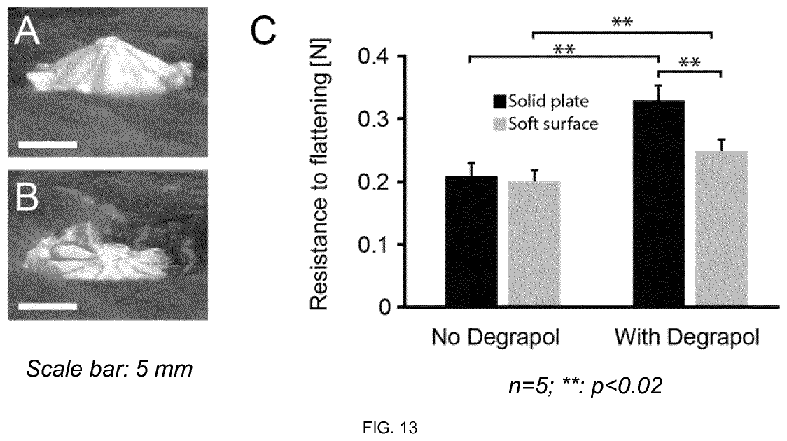

FIG. 13A shows an image of an umbrella-shaped receptor with Degrapol® membrane on porcine muscle (soft surface) before pulling test;

FIG. 13B shows an image of the umbrella-shaped receptor during the pulling test;

FIG. 13C shows histograms of the Forces needed to flatten the receptor;

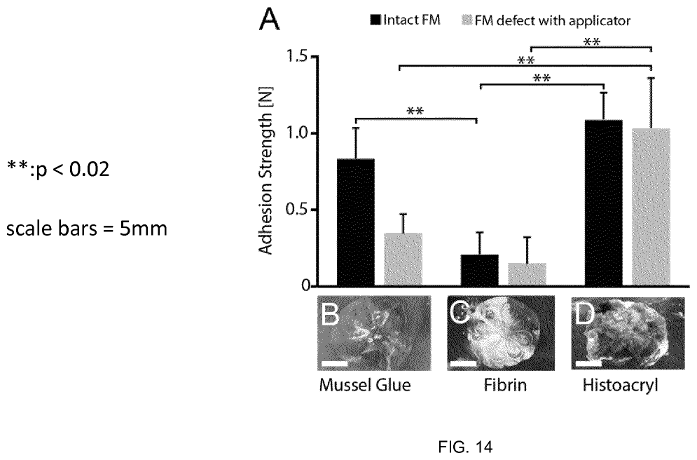

FIG. 14A shows histogram of adhesion strengths of umbrellas glued to fetal membranes;

FIG. 14B shows an image of an umbrella glued to a fetal membrane defect model with Mussel glue;

FIG. 14C shows an image of an umbrella glued to a fetal membrane defect model with Fibrin glue;

FIG. 14D shows an image of an umbrella glued to a fetal membrane defect model using Histoacryl®; and

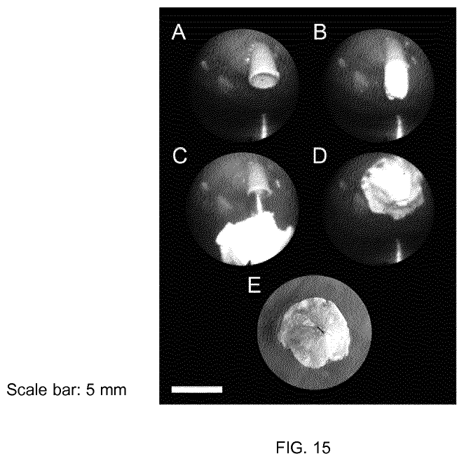

FIGS. 15A to 15D shows images that were recorded by an endoscopic camera of pulling an umbrella against a fetal membrane, followed by glue injection, release of the umbrella and removal of the applicator as well as of the catheter.

Referring to FIG. 1, a membrane 10 consisting of membrane tissue 11 is shown to enclose an amniotic cavity 20. Membrane 10 prevents fluid contained in the amniotic cavity to leak out to the membrane's surroundings 30. Membrane 10 has a perforation site 12 extending from a proximal membrane surface 14 to an inner membrane surface 16 of membrane tissue 11. Perforation site 12 can be sealed in a fluid-impermeable manner by employing a membrane closure device 100, e.g., as outlined herein below. The term “fluid” as used herein may encompass liquid, gas and/or air. In some embodiments, a patch 120 and/or a fastener 140 may be permeable to gas and/or air, but liquid-impermeable. Optionally, patch 120 and/or fastener 140 may be semi-impermeable, e.g., permeable to allow fluid to permeate from surroundings 30 into amniotic cavity 20 but impermeable for fluid to flow from amniotic cavity 20 into surroundings 30. Optionally, patch 120 and/or fastener 140 may be semi-impermeable to liquid and permeable to gas and/or air. Optionally, patch 120 and/or fastener 140 may be semi-impermeable to gas and/or air. IN some embodiments, a plurality of patches may be deployable to the same perforation site in a given procedure by membrane closure device 100, without requiring removal thereof. Optionally, membrane closure device 100 may be operable to contain and sequentially deploy a plurality of patches.

Membrane closure device 100 comprises a patch delivery mechanism 110 that comprises a tube-shaped body 111 for slidably guiding a patch 120 to perforation site 12 and, further, a fastener delivery mechanism 130 for delivering a fastener 140 to perforation site 12. In some embodiments, lubricants (e.g., oil) may be employed for facilitating the sliding of a patch 120 along the patch delivery passageway.

As exemplified in FIG. 1, patch 120 and fastener 140 may be employed for sealing perforation site 12. For example, patch 120 may be arranged in an expanded configuration (e.g., expanding latitudinally) onto an inner surface 16 of membrane 10 to cover perforation site 12, and fixed in position by a fastener 140 to seal the perforation. Otherwise stated, fastener 140 may be employed to fixedly attach or secure patch 120 in expanded configuration onto a surface of membrane 10 to seal perforation site 12. Accordingly, fastener 140 may be operative to sealantly secure patch 120 on membrane 10 to seal perforation site 12. Generally, one or more patches may be sealantly secured onto outer surface 14 and/or inner membrane surface 16 of membrane 10. For example, a first patch may be sealantly secured onto inner membrane surface 16 and a second patch may be sealantly secured to outer membrane surface 14. However, merely to simplify the discussion herein, without be construed limiting, examples and/or embodiments disclosed in conjunction with the accompanying figures refer to sealantly securing patch 120 to inner membrane surface 16.

In an embodiment, patch 120 comprises a closure sheet 121 that includes, for example, a self-growing sponge, native tissue, living tissue, Polytetrafluorethylen (PTFE), expanded Polytetrafluorethylen (ePTFE) manufactured, e.g., by employing electro-spinning; silicone-based membrane. Such silicone-based membrane may for example be coupled to structures consisting of, for example, synthetic polymer material; natural polymer material, metal; and/or any other suitable matter, or composition of matter. The silicone-based membranes may be treated, e.g., using dip-coating to produce cell-instructive membranes.

Fastener 140 may comprise, for example, native tissue, a glue and/or sealant such as, for example, a biocompatible adhesive and/or a biocompatible sealant. Exemplarily, glue that may be employed can include Mussel glue, agarose glue, fibrin glue, platelet rich plasma, thrombin-containing commercial preparations, collagen, collagen slurry, gelatine sponges, matrigel and/or any other suitable matter.

Optionally, the glue and/or sealant may have a growth factor (e.g., activation with soluble epidermal growth factor (EGF) and/or insulin-link growth factor-1 (ILGF-1); and/or comprise a cell instructive scaffold like, for example, a decellularized amniotic membrane. Optionally, the cell instructive scaffold may be glued (e.g., with fibrin), and for example loaded with mixed, soluble TGF-beta or FGF-beta.

In some embodiments, fastener 140 comprises a weld produced, for example, by a laser source.

Additional reference is made to FIGS. 2A and 2B. In an embodiment, patch delivery mechanism 110 comprises a tube-shaped body 111 having a proximal end 112 and a distal end 113 and defining internal to the body a patch delivery passageway. Patch delivery mechanism 110 may comprise a patch propelling device 114 that is adapted or configured to be employable by the user of membrane closure device 100 for pushing patch 120 through the patch delivery passageway from its proximal 112 to its distal end 113.

In an embodiment, patch propelling device 114 may comprise an elongate body 115 that can be introduced into tube-shaped body 111 and which is long enough to protrude out of the distal end of tube-shaped body 111. In an embodiment, patch propelling device 114 further comprises an actuation mechanism 116 comprising, e.g., a push button 118 and an energy storage device 119 (e.g., a mechanical energy storage device such as a compression spring) for retaining elongate body 115, and hence patch 120, by default in a retracted position so that patch is retained inside tube-shaped body 111. Energy storage device 119 is arranged and configured or adapted to retain push button 118 and hence, elongate body 115 and patch 120 thereto, by default, in the retracted position. For example, energy storage device 119 may be arranged between proximal shoulders of tube-shaped body 111 and shoulders of push button 118. Clearly, patch propelling device 114 may employ additional or alternative mechanisms for retaining patch 120 by default in the retracted position and for actuating delivery of patch 120 to perforation site 12. Patch propelling device 114 may comprise, for example, electronic, magnetic, pneumatic, hydraulic and/or any other suitable mechanism.

When the user operably engages with the handle mechanism (e.g., manually presses onto the push button), additional energy is stored by the energy storage device, elongate body 115 advances and pushes patch 120 out of tube-shaped body 111 so that patch 120 protrudes out of the tube-shaped body's distal end 113. While being pushed through tube-shaped body 111, patch 120 is in a narrow configuration. Patch 120 in the narrow configuration is schematically and exemplarily shown in FIGS. 2A and 2B in the patch-delivery passageway of tube-shaped body 111, at its distal end 113. Once patch 120 is pushed out and no longer confined within the patch delivery passageway running along tube-shaped body 111, the patch may assume the expanded configuration. When the user then releases the pressure applied onto the push button, added energy is released causing push button to retract into its initial, default position. Clearly, patch delivery mechanism may employ additional and/or alternative handle mechanisms for manually, automatically or semi-automatically propelling patch 120 including, for example, rotary knobs, levers, and/or handles.

Additional reference is made to FIGS. 2C and 2D, showing patch 120 in the expanded configuration. For instance, FIG. 2C shows patch 120 in the expanded configuration before being pushed into tube-shaped body 111, and FIG. 2D shows a more detailed view of patch 120 shown in FIG. 2C. As schematically shown in FIG. 2D, membrane closure device 100 comprises, in an embodiment, a patch manipulation arrangement, e.g., for pulling patch 120 against inner membrane surface 16 and for preventing patch 120 from falling into amniotic cavity 20, e.g., due to gravitation, after exiting distal end 113. Such patch manipulation arrangement may for example comprise a patch retention element 117A which may comprise a handhold 335 comprising a rod that runs within tube-shaped body 111. The rod of handhold 335 may protrude out of proximal slit 333 so that it can be grasped by the user. A distal end of the handhold's rod may be coupled with patch retention element 117A. Patch retention element 117A may comprise a loop to form-fittingly engage with a wire loop 1176 that is affixed to closure sheet 121. Optionally, patch retention element 117A may be suture or wire that can be used for suturing of patch 120 to membrane 10. In some embodiments, membrane closure device 100 may comprise a patch uncoupling mechanism (not shown) comprising, for example, a cutting element (not shown), for cutting wires 117A and/or 1176 open, allowing both patch delivery mechanism 110 and fastener delivery mechanism 130 be removed from perforation site 12.

Additional reference is made to FIGS. 2E and 2F. Expandable patch 120 (which may be self-expandable) further comprises a support structure 123. FIGS. 2E and 2F schematically show different embodiments of such support structure 123, respectively referenced by alphanumeric designations “123A” and “123B”.

Generally, support structure 123 is adapted or configured to support closure sheet 121. Patch 120 may be an expandable (e.g., umbrella-like) device that can be modified from a narrow to an expanded configuration. Optionally, support structure 123 is adapted or configured to permit patch 120 to be actuated to automatically expand from a narrow configuration to an expanded configuration. Expandable patch 120 may assume the expanded configuration after being propelled (e.g., pushed and/or pulled) through tube-shaped body 111 in the narrow configuration and after exiting distal end 113 for closing perforation site 12.

In some embodiments, support structure 123 stores more mechanical energy in the folded or narrow configuration than in the expanded configuration. Accordingly, once support structure 123 is no longer confined within the patch delivery passageway running along tube-shaped body 111, support structure 123 may attain the expanded configuration.

In some embodiments, support structure 123 may have to be actuated to obtain the expanded configuration. For instance, support structure 123 may be made of a shape memory material (SMA) such as nitinol, and can have a low temperature martensite state, with a martensite transition temperature below body temperature, and a comparatively high temperature austenite state at or above body temperature. Below body temperature, support structure 123 may be in its martensitic state, which is relatively soft, plastic, and easy to shape. In its austenite state, support structure 123 may assume a memorized harder material state in which support structure 123 is more difficult to deform than in the martensitic state. In some embodiments, in order for patch 120 to automatically obtain the desired shape in the expanded configuration, the materials employed for producing support structure 123 may be subjected to temperature-based shape-setting. Heating of closure sheet 121 to above body temperature where it may assume its austenite state with its memorized harder material state may in some embodiments be employed including, for example, electric charge/current flowing via a wire/electric-circuit (not shown) (possibly made of copper) or by other means such as laser light propagating via tube-shaped body 111.

According to some embodiment, support structure 123, when in the expanded configuration, may be resistant to bending forces of, for example, at least 1 Newton, 2 Newton, 5 Newton, or at least 10 Newton.

During the transition from the narrow to the expanded configuration, the support structure 123 urges closure sheet 121 to unfold.

In an embodiment, closure sheet 121 may be affixed to support structure 123. Optionally, support structure 123 may be embedded (partially or fully) in closure sheet 121, glued to closure sheet 121, and/or otherwise affixed to closure sheet 121. Optionally, in the expanded configuration, closure sheet 121 may be stretchably coupled to support structure 123.

In an embodiment, support structure 123 comprises struts that may for example be made of wires or coils.

As schematically shown in FIG. 2E, support structure 123A may for example comprise at least two wires 124A and 124B that assume, in the expanded configuration, an S-shaped form. In the expanded configuration, the at least two S-shaped wires 124A and 124B may be rotated relative to each other at their midpoints 125 such that the arcuate portions of the S-shaped wires complement a circular-like strut portion running along the outer edge of closure sheet 121. Optionally, a support structure 123 may comprise wires that assume in the expanded configuration, T-shaped, U-shaped, C-shaped, longitudinal wires and/or any other desired form.

As schematically shown in FIG. 2F, support structure 123B may comprise wires delineating in the expanded configuration the shape of a plurality of petals or lobes 126.

Generally, a petal may assume, in the extended configuration, any polygonal shape and therefore have curved and/or straight vertices and/or edges. For example, a petal 126 can have a triangular or substantially triangular shape.

In an embodiment, a wire of support structure 123B may delineate a curve resembling the shape of petalled flower or have a “Shamrock-like” shape. The curve delineated by support structure 123B may exemplarily be termed “rhodonea” or “rose curve” and may be constructed using, for example, one of the following polar equations (1) or (2):

r=αsin(nϑ) (1)

or

r=αcos(nϑ) (2)

As shown exemplarily in FIG. 2F, n=4, resulting in that the “rose” is 8-petalled. It should be noted that additional or alternative approaches or mathematical formulas can be employed to arrive at a support structure 123B delineating, e.g., a petalled contour or curve as exemplarily shown in FIG. 2F, and that the number of petals or lobes shown in FIG. 2F should by no means be construed in a limiting manner. Optionally, a petal or lobe-shaped geometry having the form of a petal 126 may be (virtually) rotated around midpoint 125 for constructing a petalled flower structure.

Further reference is made to FIG. 2G. A lobe 126, which may be made of a wire, may have the measures (in millimeters) as indicated, for example, in FIG. 2G.

Each lobe 126 may comprise two radial portions 160A and 160B virtually originating from midpoint 125 and terminating in a distal convex curve 132A having a radius R. Optionally, a lobe 126 may comprise a convex curve 132B that is proximal to midpoint 125. Optionally, two neighboring lobes 126 may be coupled with each other at adjacent radial portions 160. Optionally, adjacent radiation portions 160 of two neighboring lobes 126 may abut against each other, yet be detached from one another.

In an embodiment, at least one lobe 126 of support structure 123B may comprise coupling elements 127, e.g., for coupling support structure 123B with patch retention element 117A, which is exemplified and schematically illustrated in FIGS. 6A and 6B. Optionally, coupling element 127 may be located proximal to geometric midpoint 125 of support structure 123B. Optionally, lobe 126 may comprise a base 128 that is proximal to midpoint 125. Optionally, base 128 may comprise coupling element 127. Optionally, coupling elements 127 may be located distal to geometric midpoint 125 of a support structure, e.g., on the outer rim thereof.

Optionally, two coupling elements 127 of respective lobes 126 may be located opposite each other. Optionally, a coupling element 127 may comprise an eyelet, a hook and/or any other suitable configuration to enable the coupling of support structure 123B, and thus of patch 120, with elongate body 115 of patch propelling device 114.

Optionally, two lobes 126 that are located opposite each other may each include a coupling element 127. In embodiments where the number of lobes 126 is even, only every second lobes 126 may comprise a coupling element 127. In some embodiments, lobes 126 may have a substantially symmetrical geometry, e.g., as shown in FIGS. 2F and 2G. In some other embodiments, lobes 126 may have a non-symmetrical geometry (not shown).

Further referring to FIG. 2H, a support structure 123C may have a similar configuration as support structure 123B shown in FIG. 2F, with the optional difference that at least two or each two neighboring lobes 126 are spaced apart from each other by an angular degree, herein designated by alphanumeric reference “R”. More specifically, two juxtaposing radial portions of two neighboring lobes may define an angle of the magnitude R. Said angle R may for example range from about 5 degrees to about 25 degrees. Optionally, the angle R may for example be about 7.5 degrees or about 15 degrees. For example, the lobes of a support structure having six lobes may for example be spaced apart from each other by, e.g., 15 degrees. In another example, the lobes of a support structure having 12 lobes may for example be spaced apart from each other by, e.g., 7.5 degrees. Alternative numbers of lobes and/or angular distances than those exemplified herein may be employed for implementing a support structure.

Optionally, two radial portions of the same lobe of a patch may radially extend from a centerpoint to define an angle U ranging, for example, from about 22 to 45 degrees. Optionally, the struts may have a thickness T and V ranging, for example, from about 100 μm to about 300 μm. Optionally, a strut defining a lobe may have varying thickness. Optionally, the struts may have a thickness of about 200 μm. Optionally, the struts may have a thickness of about 150 μm. Optionally, a distance S between two coupling elements 127 may range, for example, from about 0.1 mm to about 0.3 mm. Optionally, distance S may have a magnitude of about 0.117 mm or of about 0.383 mm. Optionally, coupling elements 127 may have a diameter Q ranging, for example, from about 0.4 to about 0.5. Optionally, coupling elements 127 may have a diameter Q of about 0.556 mm. Optionally, a length W of a radial portion may range, for example, from about 3 to about 6 mm. Optionally, a radial portion may have a length W of about 3.5 mm or of about 5.282 mm. In some embodiments, the radial portion may have a length W which is suitable to accommodate a patch having in the expanded configuration a diameter of, for example, up to 30 mm. For example, the radial portion may have a length W of, e.g., at least 12 mm, or at least 15 mm.

In some embodiments, support structure 123 may be manufactured, for example, from a sheet of shape memory alloy (e.g., Nitinol) by employing laser-cutting technologies, and/or by welding and/or braiding of wires. Support structure 123 may be, in some embodiments, basket-like shaped.

Additional reference is made to FIGS. 3A and 3B, which schematically show a method of using the membrane closure device, according to some embodiments. More specifically, an example of in-vitro deployment of patch 120 by the user is schematically illustrated. FIG. 3A shows patch 120 in tube-shaped body 111 in the folded configuration, and FIG. 3B shows patch 120 in the expanded configuration.

Further reference is made to FIG. 3C. According to some embodiments, patch 120 may form, in the expanded configuration, a concave or an approximately concave surface 122 of height (H_{patch}), which may for example be defined as the distance between the rim 129 and the apex A formed by closure sheet 121 (cf. FIGS. 1 and 2D).

The patch's concave surface 122 may facilitate the gathering or holding of glue and/or sealant 140 by closure sheet 121, as is shown schematically in FIG. 1, and prevent the glue and/or sealant 140 to spill into amniotic cavity 20.

When expanded patch 120 is in an operable position, concave surface 122 may face inner membrane surface 16, thereby forming a retention cavity 150 that is adapted or configured to receive composition of matter (e.g., glue 140) that may assume or that has adhesive characteristics, so that patch 120 can adhesively couple with inner membrane surface 16, e.g., due to polymerization of the composition of matter.

In the expanded configuration, patch 120 may for example assume a height ranging from about 1.5 mm to about 5 mm. Optionally, patch 120 may assume in the expanded configuration a height of about 3 mm.

Optionally, glue 140 may assume adhesive properties only after being delivered to retention cavity 150. Optionally, glue 140 may already have adhesive properties prior to being delivered to retention cavity 150. The adhesive strength that may be imparted by glue 140 when patch 120 is adhesively coupled to membrane 10 may range, for example, from about 0.5 Newton to about 1.5 Newton, or from to about 0.5 Newton to about 5 Newton.

According to some embodiments, the volume of a cavity within fastener-delivery mechanism 130 for receiving glue 140 may correspond or be smaller than the volume of retention cavity 150 (FIG. 3C). Correspondingly, in some embodiments, fastener-delivery mechanism 130 may be configured or adapted so that the amount of glue 140 that can be delivered thereby into retention cavity 150 during one procedure does not exceed the amount of adhesive that can be received by the retention cavity. In other words, membrane closure device 100 may be operable to ensure that there is, for a given procedure, a dead volume in retention cavity 150, i.e., a certain volume of retention cavity 150 remains empty of glue 140. Thusly configured, the delivery of excess adhesive and, therefore, spilling thereof into amniotic cavity 20 may be prevented. In some embodiments, the difference between in a glue volume that can be delivered by fastener-delivery mechanism 130 and a glue volume that can be received by retention cavity 150 may be, for example, at least about 100 μl, at least about 200 μl, at least about 300 μl, at least about 400 μl, or at least about 500 μl.

Optionally, patch 120 may have to be pulled or held against inner membrane surface 16 circumferencing perforation site 12 for 5 to 180 seconds, to ensure that the patch is sealantly secured to membrane 10. After patch 120 is secured so that perforation site 12 is sealantly lidded, patch retention element 117A may be uncoupled (e.g., cut or released) by operably employing the patch uncoupling mechanism (not shown). Patch retention element 117A and wire 117B can be biocompatible and, optionally, biodegradable and/or bioabsorbable.

As already briefly outlined herein, membrane closure device 100 comprises a fastener delivery mechanism 130 for delivering a fastener 140 to perforation site 12. The fastener delivery mechanism may be manually, automatically or semi-automatically operable.

In some embodiments, a fastener delivery mechanism may be a device that is separate and independently operable from a patch propelling device. In some other embodiments, a fastener delivery mechanism may be integral to a patch propelling device. Although the discussion that follows with respect to the accompanying figures refers to embodiments where a fastener delivery mechanism is integral to a patch propelling device, this should by no means be construed limiting.

According to some embodiments, fastener delivery mechanism 130 is adapted or configured to enable controllably delivering glue 140, e.g., once patch 120 is deployed at perforation site 12 and, optionally, after it assumed the expanded configuration, for securing patch 120 with the delivered glue 140 to membrane 10. For example, fastener delivery mechanism 130 may comprise a glue injection mechanism (not shown). For example, glue injection mechanism may comprise a cavity defining internal to elongate body 115 a fastener-delivery passageway that is adapted or configured to receive glue 140. In an embodiment, the glue injection mechanism may further comprise a plunger (not shown) that is provided on the front end thereof with a piston that is slidably movable within the cavities of elongate body 115 for pressing glue 140 out of the fastener-delivery passageway.

Additional reference is made to FIGS. 4A and 4B. In some embodiments, a fastener-delivery mechanism 330 may have a Y-shaped or bifurcated configuration and may comprise at its bifurcated distal end portions a plurality of barrels (e.g., barrels 331A and 331B) of a glue injection mechanism (not shown). The plurality of barrels may converge to a shared elongate body portion 350 where they are in separate fluid communication with a plurality of fastener-delivery passageways (e.g., passageways 341A and 341B) running along elongate body portion 350 for the separate delivery and, optionally, mixing, of a corresponding plurality of glue compositions of matters (e.g., two glue components). The glue injection mechanism can for example comprise, analogous to the embodiments discussed in FIGS. 2A and 2B, a plurality of plungers (not shown) that are each provided on the front end thereof with a piston (not shown). The piston (not shown) can be slidably movable within the cavities of elongate body of barrels 331A and 331B for pressing glue 140 out of the fastener-delivery passageways.

In some embodiments, fastener delivery mechanism 330 may also function as a patch propelling device 314. Patch propelling device 314 may for example be slidably insurable into tube-shaped body 111 for pushing patch 120 outside the distal end of tube-shaped body 111.

Further reference is made to FIG. 5. In some embodiments, fastener delivery mechanism 330 may comprise a mixer 360 operative to mix two or more adhesive-components with each other for producing an adhesive. In some embodiments, elongate body portion 350 may terminate in mixer 360. Optionally, mixer 360 may be implemented as a static or dynamic mixer.

Mixer 360 may for example comprise a mixing body 361 defining therein a mixing cavity 362. Fins 363 may protrude inwardly into mixing cavity 362 for the mixing of different adhesive matter of composition. In an embodiment, fins 363 are alternatingly arranged opposite each other along the longitudinal axis of mixing body 361. In an embodiment, fins 363 are arranged in a screw-like progression along the tube-shaped mixing body.

Additional reference is made to FIGS. 6A and 6B. As already briefly outlined herein, the patch manipulation arrangement of membrane closure device 100 may for example comprise a patch retention element 117A that runs within tube-shaped body 111 and terminates at its distal end that is releasably coupled with patch 120. In one embodiment, patch retention element 117A may be a rod (not shown) that form-fittingly engages with a looped wire or thread 117B that is affixed to patch 120, like a latch or plunger. In another embodiment, patch retention element 117A may comprise a wire or thread that, optionally, runs within elongate body portion 350 and exit therefrom via a distal slit 334 for holding patch 120, and may further comprise a rod that is releasably coupled with a looped portion of the wire.

Either way, a proximal end of patch retention element 117A may be coupled to a handhold 335 that protrudes from fastener-delivery passageway, e.g., via a proximal slit 333, to allow the user to hold it for manipulation of the distal end of patch retention element 117A. FIG. 6A schematically shows patch 120 in a retained state, e.g., attached to patch retention element 117A. FIG. 6B schematically shows patch 120 in an uncoupled or released state from patch retention element 117A. According to the above noted first embodiment, patch 120 may be released by lifting the rod, exemplarily causing the thread to uncouple from elongate body portion 350. Optionally, the thread may be released from a form-fitting and/or frictional coupling state from the distal end of elongate body portion 350. According to some embodiments, upon release of the thread, a remainder portion thereof (not shown) may be received within retention cavity 150 and distal end of elongate body portion 350 may be removed in proximal direction from perforation site 12 such that the perforation extending from proximal (also: outer) membrane surface 14 to distal (also: inner) membrane surface 16 (FIG. 1) is free of or does not contain any of the following: a portion of the thread, an element of patch 120, an element of patch delivery mechanism 110, and an element of fastener delivery mechanism 130. This allows fetal membrane 10 to slide relative to the uterus (not shown) reducing the risk or without the risk of inflicting damage to either one that would otherwise be present if a stiff element were to extend through the perforation. The perforation itself may optionally be filled essentially only with glue 140.

In another embodiment, patch 120 may be coupled to the distal part of elongate body portion 350 of fastener delivery mechanism 130. Optionally, elongate body portion 350 may be slidable from a retracted to an extended position, e.g., by lifting or lowering of handhold 335 which is coupled with patch retention element 117A. In some embodiments, handhold 335 may be an extension of patch retention element 117A. Optionally, handhold 335 protrudes out of proximal slit 333.

Embodiments disclosed herein may be combined with additional wound closure techniques including, for example, cross-linking technologies and/or membrane healing through biological stimulation.

Further reference is now made to FIG. 7. As indicated by step 710, a method for closing a perforated membrane comprises, in an embodiment, deploy at least one expandable patch to a perforation site. Optionally, deploying the at least one expandable patch may include pulling or pushing the patch against the membrane surface to which the patch shall be affixed. For example, referring to FIG. 1, patch 120 may be pulled in proximal direction towards the inner surface.

As indicated by step 720, the method may further include deliver a fastener to the perforation site. As indicated by step 730, the method may include fastening the at least one expandable patch in an expanded configuration, by the fastener, to the membrane to seal the perforation.

### EXAMPLES

The present disclosure can be better understood by reference to the Example which is offered by way of illustration. The present disclosure is not limited to the Example given herein.

FIGS. 8A to 8D exemplarily show images of an example implementation of a membrane closure device. More specifically, FIG. 8A exemplarily shows an image of the elongate body or pushing cylinder of a patch propelling device responsible of pushing the patch out and retaining it for its positioning onto the uterine wall. FIG. 8B exemplarily shows an image of the membrane closure with the patch loaded inside thereof. FIG. 8C is an image exemplarily showing a more detailed view of the pushing, inner cylinder with the wire retaining the patch when deployed and an inner compartment for glue injection. FIG. 8D exemplarily shows that the transparent appearance of the patch in the folded state is due to the employment of oil that was necessary to load the patch into the tube.

FIGS. 9A to 9F show images of another example implementation of a membrane closure device. More specifically, FIG. 9A shows an image of an example inner cylinder responsible for pushing the patch out and retaining it for its positioning onto the uterine wall. FIG. 9B is an image exemplarily showing an image of two smaller tubes that extend throughout the whole construct of the pushing cylinder to deliver a sealant composed of two components. FIGS. 9C and 9D are image exemplarily showing the membrane closure device with the patch attached to it via a suture wire loop and retained by a wire controlled at the top here in its closed state. FIGS. 9E-9F exemplarily shows this same wire in its open state (patch retention element 117A is uncoupled from wire loop 117B) for releasing the patch.

FIGS. 10A and 10B show images of an example in-vitro application of the membrane closure device shown in FIGS. 8A to 8D. More specifically, FIGS. 10A and 10B show the deployment of the patch in a wet container.

FIGS. 10C and 10D show different views of the patch affixed to a wet surface with Agarose glue.

The membrane closure devices and components shown in FIGS. 8A to 10D fitted perfectly to the instruments used in clinics for fetoscopy as tested directly on catheters provided by the obstetric department. A metallic mold for strict reproducible receptor fabrication was produced and a series of umbrellas were electrospun. Their compatibility to the membrane closure devices was verified by simple insertion into it and its deployment tested in water. A test of feasibility included injecting fibrin glue through the two-chamber syringe into the concave surface of the patch. The reception capacity was confirmed by this experiment. A nitinol umbrella backbone was manufactured by McKo™. The nitinol umbrella backbone was embedded into a silicone sheet (the closure sheet). Optionally, the nitinol umbrella backbone could be coated by the electro-spun membrane.

In an additional example, a membrane closure device was employed that comprises an umbrella-shaped receptor (also: expandable patch) and an applicator (also: patch delivery mechanism). The receptor was pushed through a catheter by the applicator and expanded automatically when pushed into the amniotic cavity. The receptor was then positioned and glued against the amnion at the defect site (also: perforation site). The adhesion strength of multiple glues was tested. Additionally, the feasibility and reproducibility of this fetal membrane sealing approach was tested on an ex vivo model. The receptor opened in all tests and its positioning showed no particular difficulties (n=10). When applied via the minimal invasive procedure, receptors were glued efficiently onto the membrane and all of them completely covered the defect (n=5). The time needed for the whole procedure was slightly over 3 minutes.

The receptor for the gathering of adhesive materials, ii) the applicator to introduce the receptor (also: patch or umbrella) and iii) the injection system (also: fastener delivery mechanism) to deliver the adhesive materials inside the receptor were assembled in one single device and enabled carrying out the sealing procedure in five short steps. The idea was (1) to insert the receptor through the fetoscopy catheter, (2) deploy it inside the amniotic cavity, (3) pull it back against the inside wall of the fetal membrane and (4) glue the umbrella onto the fetal membrane. (5) The catheter is then removed while the umbrella is left glued to the membrane until it comes out with the baby's birth.

The Applicator shown in is responsible to push the Umbrella inside the 10 French introducer (not shown). The Glue injection mechanism (also: fastener delivery mechanism) is the entry site for the glue, which was delivered to the Umbrella with the assistance of a double chambered tube placed inside the Applicator. A release mechanism of the Umbrella is controlled by one small metallic wire going through the Applicator (Lock). When in the Locked position, the wire traps the suture wire connected to the umbrella. When in the unlocked position, the suture wire is no longer trapped and the umbrella can be unconnected from the Applicator.

Components that were used in the Experiments for the Preventive Sealing of Fetal Membranes

Umbrella-Shaped Receptor for the Gathering of Sealing Materials:

Reference is made to FIGS. 11A-11B. The receptor comprised two elements: a nitinol backbone and a membrane covering that backbone in order to gather the adhesives. The nitinol stripes thickness was set at 200 μm and the umbrella-shaped height at 3 mm. The receptor was composed of 8 identical oval struts each connected to the neighbouring one, forming a circular shape. The connection of all small part of each oval formed a small circle with a diameter smaller than the 10 French catheter at the proximal part of the umbrella (FIG. 11A). They were produced by laser cutting followed by shape setting.

Degrapol® was chosen because of its in-house production availability and its similar stretchability. In addition, it has previously shown a capacity to integrate into rabbit fetal membranes and promote reepithelialisation. (Ochsenbein-Kolble, N., et al. Enhancing sealing of fetal membrane defects using tissue engineered native amniotic scaffolds in the rabbit model. American journal of obstetrics and gynecology 196, 263 e261-267 (2007).

To produce those sheets, 600 mg of lyophilized Degrapol® was dissolved in 3.52 g of Chloroform and 0.88 g of Hexafluoroisopropanol to make 5 g of 12% Degrapol® mix. The solution was left at room temperature overnight and 2 ml of this solution was electro-spun at a rate of 1 ml per hour on 12 glass slides rotating on the collector. The distance between the syringe needle and the collector was 10 cm. The resulting Degrapol® sheets of approximately 150 μm thickness were removed from the glass slides by immersing in 30% ethanol. The nitinol backbone was inserted between two Degrapol® sheets that were pressed together to fuse them.

Applicator and Injector

Further reference is made to FIG. 11C. A stainless-steel tube with an outer diameter of 2.9 mm and inner diameter of 2.7 mm was designed and manufactured. This tube slid freely in the 10 French catheter and was tight enough to push the receptor (FIG. 11C, “Receptor”) through the catheter. A double chamber injector (Duplocath 180, Baxter AG, Volketswil, Switzerland) from the Tisseel® glue pack was inserted into the hollow tube from the proximal to the distal end to ensure that the two glue substrates only merge at the distal end of the tube into the receptor. The proximal end consisted of two entry sites for two syringes, each for one component of a two precursors glue.

The receptor had to remain attached to the applicator in order to prevent the loss of it in the amniotic cavity upon deployment and to allow the pulling against the fetal membrane defect. To do so, a thread was attached to the umbrella-shaped receptor and connected it to a locking system at the distal part of the applicator. Upon unlocking of this system by the user, the thread was set free and the receptor was detached from the applicator.

Bioadhesives:

To test the fetal membrane sealing procedure Histoacryl® (B.Braun Surgical AG, Melsungen, Switzerland), fibrin glue (Tisseel®, Baxter AG, Volketswil, Switzerland), and mussel glue were employed (Brubaker, C. E., Kissler, H., Wang, L. J., Kaufman, D. B. & Messersmith, P. B. Biological performance of mussel-inspired adhesive in extrahepatic islet transplantation. Biomaterials 31, 420-427 (2010); Burke, S. A., Ritter-Jones, M., Lee, B. P. & Messersmith, P. B. Thermal gelation and tissue adhesion of biomimetic hydrogels. Biomedical materials 2, 203-210 (2007); Lee, B. P., Dalsin, J. L. & Messersmith, P. B. Synthesis and gelation of DOPA-modified poly(ethylene glycol) hydrogels. Biomacromolecules 3, 1038-1047 (2002); Lee, H., Lee, B. P. & Messersmith, P. B. A reversible wet/dry adhesive inspired by mussels and geckos. Nature 448, 338-341 (2007); Lee, H., Scherer, N. F. & Messersmith, P. B. Single-molecule mechanics of mussel adhesion. Proc Natl Acad Sci USA 103, 12999-13003 (2006).

Histoacryl® is a cyanoacrylate-based, Federal Drug Administration (FDA) approved one component glue that polymerizes upon contact with hydroxyl ions present in wet environments. Due to its strong gluing ability, it is used for application such as net fixation for inguinal hernia.27 Histoacryl® was directly transferred from its ampoule to a syringe tube before application. Fibrin is another commonly used tissue sealant that showed potentially good binding ability to fetal membranes.21 Fibrin glue is composed of two precursors, Sealer Protein (human plasma) and Thrombin, which are injected simultaneously inside the two-chamber injector tube and polymerize instantaneously when gathered in the receptor. The mechanism of action replicates the formation of blood clot in the vascular system where thrombin activates the coagulation cascade.

Mussel glue is a sealant that readily polymerizes upon mixing of the polymer precursor with its activator and was shown to have very good properties for the sealing fetal membranes under wet conditions.20 As previously described the polymer substrate is dissolved in 2×PBS and mixed in a 1:1 volume ratio to 12 mg/ml sodium periodate for a final mussel glue concentration of 150 mg/ml.

Human Fetal Membranes

Human fetal membranes were collected from patients with written consent following the decision from the Ethical Committee of the District of Zürich (study Stv22/2006). Fetal membranes were harvested after caeserian section at term (between 37 and 39 weeks) and frozen at −80° C. until usage. All of them were negatively selected for HIV, hepatitis A and B, diabetes mellitus, chlamydia, cytomegaly and streptococcus B.

Mechanical Testing

Mechanical Stability of the Receptor

The mechanical stability of the receptor was assessed by measuring the force needed to completely flatten the receptor. A thread was attached to the center culm of the receptor with its hollow part facing down. The thread was then pulled until the receptor was flattened and the force needed to do so was measured with a dynamometer. Receptors with and without Degrapol® membrane were pulled on solid (Petri dish) and soft surfaces (1.5 cm thick pork muscle).

Adhesion Strength provided by Bioadhesives to Glue the Receptor to Intact Fetal Membranes

Intact fetal membranes were flattened on a Petri dish with the amnion facing up. The receptors were placed on top of the membrane followed by the injection of 500 μl of glue inside the receptors through a syringe and by 5 minutes of polymerization time. The adhesion strength provided by the glues was measured with a dynamometer (Pesola AG, Schindellegi, Switzerland, ref.DO29/FH27 by pulling the receptors perpendicularly to the membrane surface.

Application and Gluing of Receptor on Punctured Fetal Membranes

Dry Experimental Conditions

A fetal membrane was mounted onto a plastic cup with the amnion facing downward to the interior of the cup. A very high bonding elastomeric membrane (3M AG, Rüschlikon, Switzerland) was punched with a 6 mm diameter biopsy punch and deposited onto the fetal membrane. A sharp obturator (Anklin AG, Switzerland) was used to punch through the fetal membrane to create the defect. A 10 French catheter (Check-Flo Performer® Introducer, Cook Medical, Bloomington, Ind., USA) was introduced in the defect and the receptor was introduced through it into the interior of the cup. The receptor was held for 5 minutes against the fetal membrane following injection of Mussel Glue, Fibrin or Histoacryl®. The membranes were unmounted from the plastic cup and turned upside down. The umbrella was pulled perpendicularly to the membrane surface and the force needed to remove the receptor from the membrane was measured with a dynamometer.

Complete Ex Vivo Fetal Membrane Defect Model

Reference is now made to FIGS. 12A-12C. FIG. 12A shows a side view image of an ex vivo fetal membrane mode clamped in an aluminum cylinder; FIG. 12B shows a top view image of the tissue clamped in the aluminum cylinder; and FIG. 12C shows a bottom view of the tissue clamped in the aluminum cylinder. Porcine skin containing a small muscle layer with a total thickness of 0.5 cm was chosen to mimic the upper abdominal wall. A porcine muscle of 1 cm thickness and a human fetal membrane were added under the abdominal wall mimic to reproduce the uterus layer. Those three layers were put together, stretched, placed onto a 8 cm diameter aluminum cylinder and tightly clamped by tightly screwing a ring on top. The construct has a transparent bottom for visualization purposes and was water tight. Two inlets in the cylinder enabled the cylinder to fill with solutions reproducing the amniotic fluid. Another inlet permitted the insertion of an endoscopic camera in order to have a visual feedback of the interior of the cavity.

A 5 mm cut was made with a scalpel on the skin mimic to facilitate the introduction of a sharp obturator (Anklin AG, Switzerland), which was used to punch through the abdominal and uterine wall mimic as well as through the fetal membrane. A 10 French catheter (Check-Flo Performer® Introducer, Cook Medical, Bloomington, Ind., USA) used in fetoscopic interventions was introduced at the same time by mounting it on obturator. The receptor was crimped and inserted in the catheter before pushing it inside the cavity filled with saline solution. The receptor was pulled against the membrane and 500 μl of glue (100 μl corresponding to the dead volume of the double-chamber injection channel plus 400 μl adhesive) was perfused through the injector into the receptor using syringes connected to the injection inlets. Polymerization was allowed for 1 minute while the receptor was held against the fetal membrane in its umbrella-shaped conformation. A video-intubation system (Acutronice Medical Systems AG, Switzerland) enabled the visual control over the placement of the receptor. A total of 4 defects were sealed on one model. After the procedure membranes were released from the cylinder clamp and unmounted from the plastic cup or cylinder devices and turned upside down for further evaluations.

Results:

As the central part of the strategy explained here, the receptor was designed such that it fits through the catheter, deploys into a larger, predefined shape, gathers the adhesive at the site of defect and remains glued to the amnion until delivery. A circular structure was adopted, which structure was made of Nitinol, which enables the crimping into the catheter and spontaneous deployment into the original shape upon exit of the catheter. This metallic backbone was integrated into an elastic Degrapol® membrane to confine the adhesives inside the receptor and limit the exchange with the amniotic fluid. Additionally, to enable the user to feel a resistance before its flattening or even everting, the receptor was given a curvature of 3 mm. The applicator, by which the receptor is pushed through the catheter into the amniotic cavity, was made out of a tube with an outer diameter (3 mm) and a length exceeding the catheter's size by 10 mm. The applicator was shown to slide through the 10 French catheter with a minimal force (0.2N±0.02N) and access the inside of the amniotic cavity.

The resistance of receptors with or without Degrapol® was compared against each other to evaluate the contribution of the membrane to the receptor's stability. In addition, an assay was performed on a plastic Petri dish or on a 1.5 cm thick muscle layer, which assay was aimed at determining the impact of the support on which the receptor is measured.

Reference is made to FIGS. 13A to 13C. FIG. 13A shows an image of an umbrella-shaped receptor with Degrapol® membrane on porcine muscle (soft surface) before pulling test. FIG. 13B shows an image of the umbrella-shaped receptor during the pulling test. FIG. 13C shows histograms of the Forces needed to flatten the receptor.

While the backbone on the stiff and the soft surface deformed at almost identical forces of 0.21±0.02 N and 0.2±0.02 N, respectively, the resistance to deformation of the full umbrella was significantly increased on both soft and stiff substrates. The force for flattening was significantly higher on the solid substrate (0.33±0.02N) as compared to the soft substrate (0.25±0.02N). This indicates that the receptor needs to be tested with the membrane on a soft tissue to ensure the relevance of the assay for its in vivo application.

Adhesion Strength of the Receptor to Intact Fetal Membranes

Additional reference is made to FIGS. 14A to 14D. FIG. 14A shows histogram of adhesion strengths of umbrellas glued to fetal membranes. FIG. 14B shows an image of an umbrella glued to a fetal membrane defect model with Mussel glue. FIG. 14C shows an image of an umbrella glued to a fetal membrane defect model with Fibrin glue. FIG. 14D shows an image of an umbrella glued to a fetal membrane defect model using Histoacryl®. The black bars of the histogram illustration in FIG. 14A represent the cases where the receptors were directly glued on intact fetal membranes. Grey bars represent the cases when the receptors were applied through the defect and glued on the fetal membrane under dry conditions.

In the first step, we evaluated the potential of three adhesive candidates to glue the Degrapol® coated receptors to the fetal membrane. For this the receptor was deposited on intact fetal membranes and the adhesive was injected in the receptor using a syringe. After 5 minutes of polymerization, the force to detach the receptor from the membrane was measured. While mussel glue reached similar adhesion forces as Histoacryl® (0.83±0.2N and 1.1±0.17 N respectively, n=5), the adhesion of Fibrin with 0.2±0.14N (n=5) was very weak (FIG. 14A, black bars). As a drawback, Histoacryl® had the effect of drying out both fetal and the Degrapol® membranes.

Application and Gluing of the Receptor on Fetal Membrane Defects under Dry Experimental Conditions

Next, we assessed the ability of the receptor to be pushed through the catheter, to deploy, to be positioned and glued on the fetal membrane. This was done on a model consisting of a punctured fetal membrane and a transparent elastomeric membrane in dry experimental conditions. In all the trials (n=13), the umbrellas deployed to their initial shape, were placed tightly on the fetal membrane and no leakage of the glue was observed upon injection. In addition, all the receptors adhered to the fetal membrane following adhesion and release. However, all the glues showed different values of adhesion strength. Histoacryl® resisted up to 0.96±0.38N (n=3) of pulling force until detachment, compared to 0.35±0.12N (n=5) and 0.17±0.18N (n=5) for Mussel glue and Fibrin respectively (FIG. 14A, grey bars).

Fetal Membrane Defect Ssealing Procedure on Complete Ex Vivo Model

To conclude the proof of feasibility of the approach, a physiologically relevant model was created for the simulation of the catheterization and the sealing of the fetal membrane with our device. An ex vivo model was established to mimic the abdominal, the uterus and fetal membrane layers by the assembly of porcine skin containing a small muscle porcine muscle and a human fetal membrane. The resulting closed cavity was then filled with saline solution, which mimicked the amniotic fluid and provided a more realistic value to the ex vivo model. The tissue mimic had a thickness comparable to the human morphology and besides conferring a physiologically more relevant model also had a higher mechanical stability, which contributes to the resistance strength when pulling the receptor against the membrane. To quantify the mechanical stability, the force needed to pull up the center of the model by 1.5 cm was measured. The ex vivo model required 1 N for this displacement, compared to 0.1 N in the model where the fetal membrane was only supported by an elastomeric membrane.

Defect Sealing Ex Vivo

Mussel Glue was taken as the adhesive of choice to be tested in the ex vivo model. A 5 mm incision was made on the skin to facilitate punching through the tissue layers. The 10 French catheter was mounted on the obturator and inserted through the tissue mimic in a 45° angle. The obturator was removed and the catheter left in the defect. The receptor was crimped, inserted in the catheter and pushed with the applicator until deployment inside the cavity. The umbrella was pulled against the fetal membrane, followed by glue injection, release of the umbrella and removal of the applicator as well as the catheter. The procedure was recorded by an endoscopic camera, which steps of the procedure are shown in the images of FIGS. 15A to 15E.

More specifically, FIGS. 15A to 15E illustrate the procedure from inside of the cylinder, reproducing the view from the amniotic cavity, wherein FIG. 15A shows accession of the catheter (blue tube) right after punching through the construct to create the defect, in FIG. 15B the white element shows the umbrella in its crimped comformation being introduced, FIG. 15C, when deployed and FIG. 15D when pulled against the fetal membrane. FIG. 15E shows an image when the Mussel Glue was absorbed by the Degrapol®, changing its color to red. The figure also shows the umbrella glued onto the fetal membrane after dismantling.

In all trials (n=5), the receptor opened into its original shape and was properly placed onto the defect. The glue was well confined in the umbrella despite a few observations of minimal leakage. These were mainly due to the slight permeability of the Degrapol® but it never impaired the adhesion of the receptor to the fetal membranes. Indeed, all the receptors held onto the fetal membranes upon release and removal of the applicator. The strength at which they were glued to the membrane was then evaluated the same way as described in the two previous models. On average, 0.13±0.04N were needed to detach the receptor from the fetal membranes.

Time of Procedure

The time needed for each step of the procedure were reported, which can be a potential limitation for the adoption of our method. Those time intervals were summarized in Table 1. Device application, which included crimping of the receptor and placement of it onto the fetal membrane, was the longest step of the procedure with an average of 1 minute and 28±43 seconds. Glue injection time had a constant 1 minute polymerization period in all the tests, to which 27±7 seconds were needed to place the syringes on the glue injection inlets and apply the glue. Finally, 13±9 seconds were added to activate the locking system and release the receptor. In summary, the total average time for the whole procedure was 3 minutes and 08 seconds, which represents an insignificant increase of time for fetoscopy.

TABLE 1
Statistics of the fetal membrane sealing procedure on an ex vivo model.

| | Time | | |
|---|---|---|---|
| | [min:sec] | Comment | Success rate |
| Device application | 1:28 ± 0 :43 | Umbrella deployed and was placed | 5/5 (100%) |
| | | on the defect in every test. | |
| Glue injection | 1:27 ± 0:07 | Injection was straight forward. | 5/5 (100%) |
| | | Some leakage through Degrapol ® | |
| | | was observed due to the relative | |
| | | permeability of this material, | |
| | | without hindering gluing capacity. | |
| Umbrella release | 0:13 ± 0:09 | All the umbrellas remained attached | 5/5 (100%) |
| | | to the membrane upon release. | |
| Adhesive forces of glued | 0.13 ± 0.04 | | |
| umbrellas [N] | | | |

Discussion of the Experiments:

The experiments proved the feasibility of our method for the precise application of tissue sealants at the site of fetal membrane defect. To this end, the model that was developed reproduced close to real anatomic conditions to enable the realistic simulation of the procedure ex vivo. As for the method to seal the defect a device was designed and produced that facilitates the precise deposition of bioadhesives at the ruptured site, which is believed to be a significant reason for the limited success of previous attempts to seal the membrane.

The deliberate choice of diverse sealing materials—two-component glues like fibrin or the mussel glue and Histoacryl®—was done to demonstrate the compatibility of our method to materials of different natures and to highlight the relevance of our method for the testing of those injectable candidates.

### Additional Examples

Example 1 includes a membrane closure device for closing a perforation in a membrane, comprising: a patch delivery mechanism for guiding at least one expandable patch to a perforation site of a membrane; and a fastener delivery mechanism for delivering a fastener to the perforation site and for fastening the at least one expandable patch by the fastener to the membrane to seal the perforation in the membrane by the at least one expandable patch.

Example 2 includes the subject matter of example 1 and, optionally, at least one tube or tube-shaped body having a distal and a proximal end for delivering the at least one expandable patch in a folded configuration via the tube from the distal to the proximal end for attaching the at least one expandable patch in an expanded configuration to an inner surface of the membrane.

Example 3 includes the subject matter of example 1 or 2 and, optionally, wherein the at least one expandable patch is a self-expandable patch.

Example 4 includes the subject matter of example 3 and, optionally, wherein the self-expandable patch is an umbrella-like device comprising struts and a closure sheet affixed to the struts.

Example 5 includes the subject matter of example 4 and, optionally, wherein the struts comprise at least two S-shaped wires.

Example 6 includes the subject matter of example 5 and, optionally, wherein the struts comprise shape-memory material composed of, for example, Nitinol.

Example 7 includes the subject matter of any one of examples 1 to 6 and, optionally, wherein the at least one expandable patch, in the expanded configuration, has a concave surface facing the membrane for the reception of glue.

Example 8 includes the subject matter of any one of examples 2 to 7 and, optionally, wherein the fastener delivery mechanism comprises the at least one tube for delivering the fastener to the perforation site.

Example 9 includes the subject matter of any one of the examples 2 to 7 and, optionally, wherein the fastener delivery mechanism comprises at least one other tube, different from the at least one tube, for delivering the fastener to the perforation site.

Example 10 includes the subject matter of any one of the examples 1 to 9 and, optionally, wherein the fastener comprises any one of the following: biocompatible adhesive, biocompatible sealant, sealant-inducing material, and/or weld.

Example 11 includes the subject matter of any one of the examples 1 to 10 and, optionally, wherein the fastener delivery mechanism comprises an energy source such as, for example, a laser source.

Example 12 includes the subject matter of any one of the examples 1 to 11 and, optionally, wherein the fastener delivery mechanism comprises an adhesive mixing chamber.

Example 13 includes the subject matter of example 12 and, optionally, wherein the adhesive mixing chamber comprises a tube-shaped body and fins, wherein the fins protrude inwardly into the tube-shaped body for the mixing of different adhesive matter of composition.

Example 14 includes the subject matter of example 13 and, optionally, wherein the fins are alternatingly arranged opposite each other along the tube's longitudinal axis.

Example 15 includes the subject matter of examples 13 or 14 and, optionally, wherein the fins are arranged in a screw-like progression along the tube-shaped body.

Example 16 includes the subject matter of any one of the examples 1 to 15 and, optionally, comprising a patch uncoupling mechanism for releasing or cutting the at least one expandable patch from the membrane closure device following fastening of the at least one expandable patch to the inner membrane surface of the perforation site of the membrane.

Example 17 includes the subject matter of any one of the examples 1 to 16 and, optionally, wherein the fastener comprises any one of the following: a growth factor and/or a cell instructive scaffold like, for example, a decellularized amniotic membrane.

Example 18 includes the membrane closure device according to any of the example 1 to 17 for use in treating a perforation of a fetal membrane, for use in treating a perforation of a vascular membrane, for use in treating a perforation of a cardiovascular membrane and/or for use in treating a tympanic membrane.

Example 19 includes a method for closing a perforation in a membrane by a membrane closure device, comprising:

delivering at least one expandable patch to a perforation site; delivering a fastener to the perforation site; and fastening the at least one expandable patch by the fastener in an expanded configuration to the membrane to seal the perforation.

Example 20 includes the subject matter of example 19 and, optionally, operably deploying a plurality of patches.

Example 21 concerns a membrane closure device for closing a perforation in a membrane having a proximal side and a distal side, the membrane closure device comprising: a patch that is selectively expandable from a narrow to an expanded configuration and having in the expanded configuration a concave surface defining a retention cavity for receiving glue; a patch delivery mechanism for guiding the patch through a perforation of the membrane from a proximal to a distal side thereof; a fastener delivery mechanism operable to contain glue and further operable to deliver the glue contained therein to from the proximal side to the distal side of the membrane for fastening the patch in the expanded configuration, by the glue, to the distal side of the membrane to seal the perforation.

Example 22 includes the subject matter of example 21 and, optionally, wherein the retention cavity defines a volume that is larger than a volume of glue that can be contained, at any given time, by the fastener mechanism.

Example 23 includes the subject matter of example 21 or example 22 and, optionally, at least one tube having a distal and a proximal end for delivering the expandable patch in a folded configuration via the tube from the distal to the proximal end for attaching the expandable patch in an expanded configuration to an inner surface of the membrane.

Example 24 includes the subject matter of any one of the examples 21 to 23, wherein the expandable patch is a self-expandable patch.

Example 25 includes the subject matter of example 24 and, optionally, wherein the self-expandable patch is an umbrella-like device comprising a support structure including struts and a closure sheet affixed to the struts.

Example 26 includes the subject matter of example 25 and, optionally, wherein the struts comprise at least two wires that assume, in the expanded configuration, an S-shaped form or the form or contour of a petalled flower.

Example 27 includes the subject matter of example 26 and, optionally, wherein the struts comprise shape-memory material composed of a shape memory alloy such as, for example, Nitinol.

Example 28 includes the subject matter of any one of the examples 21 to 27 and, optionally, wherein the glue comprises a biocompatible adhesive, a biocompatible sealant, a sealant-inducing material, and/or a healing-inducing material.

Example 29 includes the subject matter of any one of the examples 21 to 28 and, optionally, wherein the fastener delivery mechanism comprises a mixer that is included in the tube-shaped body for the mixing of components to produce an adhesive.

Example 30 includes the subject matter of example 29 and, optionally, wherein the mixer comprises a mixing body and fins, wherein the fins protrude inwardly into the mixing body for the mixing of different adhesive matter of composition.

Example 31 includes the subject matter of any one of the examples 21 to 30 and, optionally, a patch uncoupling mechanism for releasing the at least one expandable patch from the membrane closure device following fastening of the expandable patch to the inner membrane surface of a perforation site of the membrane.

Example 32 includes the subject matter of any one of the examples 21 to 31 and, optionally, wherein the fastener comprises any one of the following: a growth factor and/or a cell instructive scaffold like, for example, a decellularized amniotic membrane.

Example 33 includes the subject matter of any one of the examples 21 to 32 for use in treating a perforation of a fetal membrane, for use in treating a perforation of a vascular membrane, for use in treating a perforation of a cardiovascular membrane and/or for use in treating a tympanic membrane.

Example 34 concerns a method for closing a perforation in a membrane by a membrane closure device, the method comprising: delivering an expandable patch to a perforation site; delivering a fastener to the perforation site; and fastening the expandable patch by the fastener in an expanded configuration to the membrane to seal the perforation.

Example 35 includes the subject matter of example 34 and, optionally, operably deploying a plurality of patches.

Unless otherwise stated, the use of the expression “and/or” between the last two members of a list of options for selection indicates that a selection of one or more of the listed options is appropriate and may be made.

In the claims or specification of the present application, unless otherwise stated, adjectives such as “substantially” and “about” modifying a condition or relationship characteristic of a feature or features of an embodiment of the invention, are understood to mean that the condition or characteristic is defined within tolerances that are acceptable for operation of the embodiment for an application for which it is intended.

It should be understood that where the claims or specification refer to “a” or “an” element and/or feature, such reference is not to be construed as there being only one of that element and/or feature.

Terms used in the singular shall also include the plural, except where expressly otherwise stated or where the context otherwise requires.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

The various features and steps discussed above can be mixed and matched by one of ordinary skill in the art in accordance with principles described herein.

Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

In the description, unless otherwise stated, adjectives such as “substantially” and “about” that modify a condition or relationship characteristic of a feature or features of an embodiment of the invention, are to be understood to mean that the condition or characteristic is defined within tolerances that are acceptable for operation of the embodiment for an application for which it is intended.

It is appreciated that certain features of the invention, which are, for clarity, described in the context of separate embodiments or examples, may also be provided in combination in a single embodiment. Conversely, various features of the invention, which are, for brevity, described in the context of a single embodiment, may also be provided separately or in any suitable sub-combination or, as suitable, in any other described embodiment of the invention. Certain features described in the context of various embodiments are not to be considered essential features of those embodiments, unless the embodiment is inoperative without those elements.

It is important to note that methods discussed herein are not limited to those diagrams or to the corresponding descriptions. For example, the methods may include additional or even fewer processes or operations in comparison to what is described in the figures. In addition, embodiments of the method are not necessarily limited to the chronological order as illustrated and described herein.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise specified, the terms ‘about’ and/or ‘close’ with respect to a magnitude or a numerical value may imply to be within an inclusive range of −10% to +10% of the respective magnitude or value.

It is noted that the terms “operable to” can encompass the meaning of the term “adapted or configured to”. In other words, a machine for example that is “operable to” perform a task can in some embodiments, embrace a mere capability (e.g., “adapted”) to perform the function and, in some other embodiments, a machine that is actually made (e.g., “configured”) to perform the function.

As used herein, the phrase “A, B, C, or any combination of the aforesaid” should be interpreted as meaning all of the following: (i) A or B or C or any combination of A, B, and C, (ii) at least one of A, B, and C; and (iii) A, and/or B and/or C. This concept is illustrated for three elements (i.e., A, B, C), but extends to fewer and greater numbers of elements (e.g., A, B, C, D, etc.).

While the invention has been described with respect to a limited number of embodiments, these should not be construed as limitations on the scope of the invention, but rather as exemplifications of some of the embodiments.

1. A membrane closure device (100) for closing a perforation in a membrane (10) having a proximal side and a distal side, the membrane closure device (100) comprising:
a patch (120) that is selectively expandable from a narrow to an expanded configuration and having in the expanded configuration a concave surface (122) defining a retention cavity (150) for receiving glue (140);
a patch delivery mechanism (110) for guiding the patch (120) through a perforation of the membrane (10) from a proximal to a distal side thereof;
a fastener delivery mechanism (130, 330) operable to contain glue (140) and further operable to deliver the glue (140) contained therein from the proximal side to the distal side of the membrane (10) for fastening the patch (120) in the expanded configuration, by the glue (140), to the distal side of the membrane (10) to seal the perforation.

2. The membrane closure device (100) of claim 1, wherein the retention cavity (150) defines a volume that is larger than a volume of glue (140) that can be contained, at any given time, by the fastener delivery mechanism (130, 330).

3. The membrane closure device (100) according to claim 1, comprising at least one tube having a distal and a proximal end for delivering the patch (120) in a folded configuration via the at least one tube from the distal to the proximal end for attaching the patch (120) in an expanded configuration to an inner surface (16) of the membrane (10).

4. The membrane closure device (100) according to claim 1, wherein the patch (120) is a self-expandable patch (120).

5. The membrane closure device (100) according to claim 4, wherein the self-expandable patch (120) is an umbrella-like device comprising a support structure (123A, 123B) including struts and a closure sheet (121) affixed to the struts.

6. The membrane closure device (100) according to claim 5, wherein the struts comprise at least two wires (124A, 124B) that assume, in the expanded configuration, an S-shaped form or the form or contour of a petalled flower.

7. The membrane closure device (100) according to claim 6, wherein the struts comprise shape-memory material composed of, for example, Nitinol.

8. The membrane closure device (100) according to claim 1, wherein the glue (140) comprises a biocompatible adhesive, a biocompatible sealant, a sealant- inducing material, and/or a healing-inducing material.

9. The membrane closure device (100) according to claim 1, wherein the fastener delivery mechanism (130, 330) comprises a mixer (360) that is included in a tube-shaped body (111) of the device for mixing of components to produce an adhesive.

10. The membrane closure device (100) according to claim 9, wherein the mixer (360) comprises a mixing body (361) and fins (363), wherein the fins (363) protrude inwardly into the mixing body (361) for mixing of different adhesive matter of composition.

11. The membrane closure device (100) according to claim 1, comprising a patch uncoupling mechanism for releasing the patch (120) from the membrane closure device following fastening of the patch (120) to the inner surface (16) of a perforation site (12) of the membrane (10).

12. The membrane closure device (100) according to claim 1, wherein the glue (140) comprises any one of the following: a growth factor and/or a cell instructive scaffold like, for example, a decellularized amniotic membrane.

13. The membrane closure device (100) according to claim 1, for use in treating a perforation of a fetal membrane, for use in treating a perforation of a vascular membrane, for use in treating a perforation of a cardiovascular membrane and/or for use in treating a tympanic membrane.

14. The membrane closure device (100) according to claim 1, wherein the patch, when affixed to the membrane, is free of mechanical fastener elements requiring perforation and/or puncturing of the membrane.

15. A method for closing a perforation in a membrane (10) by a membrane closure device (100), comprising:
delivering a patch (120) to a perforation site (12);
delivering a glue (140) to the perforation site (12); and
fastening the patch (120) by the glue (140) in an expanded configuration to the membrane (10) to seal the perforation.

16. The method of claim 15 comprising operably deploying a plurality of patches (120).
